(19) United States
(12) Patent Application Publication
(10) Pub. No.: US 2018/0318542 A1
(43) Pub. Date: Nov. 8, 2018

(54) RETRACTABLE TUBING DEVICE

(71) Applicant: Patricia Knowlson, Washington, PA (US)

(72) Inventor: Patricia Knowlson, Washington, PA (US)

(21) Appl. No.: 15/584,095

(22) Filed: May 2, 2017

(51) Int. Cl. A61M 16/08; B65H 75/28; B65H 75/40; B65H 75/44; B65H 75/48; A61M 16/06; A61M 16/10

(52) U.S. Cl. CPC A61M 16/0875; B65H 75/28; B65H 75/406; B65H 75/4478; B65H 75/486; A61M 2209/082; A61M 16/0666; A61M 16/101; A61M 2209/088; A61M 2202/0208; A61M 16/0816

(57) ABSTRACT

A retractable tubing device for supplying oxygen to a user includes a housing. A cylinder is positioned in and rotationally coupled to the housing. The cylinder extends between a first side and a second side of the housing to define a chamber. A nozzle is coupled to the housing and is fluidically coupled to the chamber. The nozzle is configured to couple to tubing that is fluidically coupled to an oxygen supplying interface. A tube, which is flexible, has a first end that is fluidically coupled to the cylinder. The tube extends through an opening positioned in the housing. The tube is biased to a retracted configuration wherein the tube is coiledly positioned around the cylinder. The cylinder is positioned to rotate relative to the housing. The tube is selectively and reversibly extensible from the housing so that the tube is configured to couple to a source of oxygen.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to retractable tubing devices and more particularly pertains to a new retractable tubing device for supplying oxygen to a user.

### BRIEF SUMMARY OF THE INVENTION

An embodiment of the disclosure meets the needs presented above by generally comprising a housing. A cylinder is positioned in and rotationally coupled to the housing. The cylinder extends between a first side and a second side of the housing to define a chamber. A nozzle is coupled to the housing and is fluidically coupled to the chamber. The nozzle is configured to couple to tubing that is fluidically coupled to an oxygen supplying interface. A tube, which is flexible, has a first end that is fluidically coupled to the cylinder. The tube extends through an opening positioned in the housing. The tube is biased to a retracted configuration wherein the tube is coiledly positioned around the cylinder. The cylinder is positioned to rotate relative to the housing. The tube is selectively and reversibly extensible from the housing so that the tube is configured to couple to a source of oxygen.

There has thus been outlined, rather broadly, the more important features of the disclosure in order that the detailed description thereof that follows may be better understood, and in order that the present contribution to the art may be better appreciated.

There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a retractable tubing device according to an embodiment of the disclosure.

FIG. 2 is a side view of an embodiment of the disclosure.

FIG. 3 is a back view of an embodiment of the disclosure.

FIG. 4 is a cross-sectional view of an embodiment of the disclosure.

FIG. 5 is an in-use view of an embodiment of the disclosure.

### DETAILED DESCRIPTION OF THE INVENTION

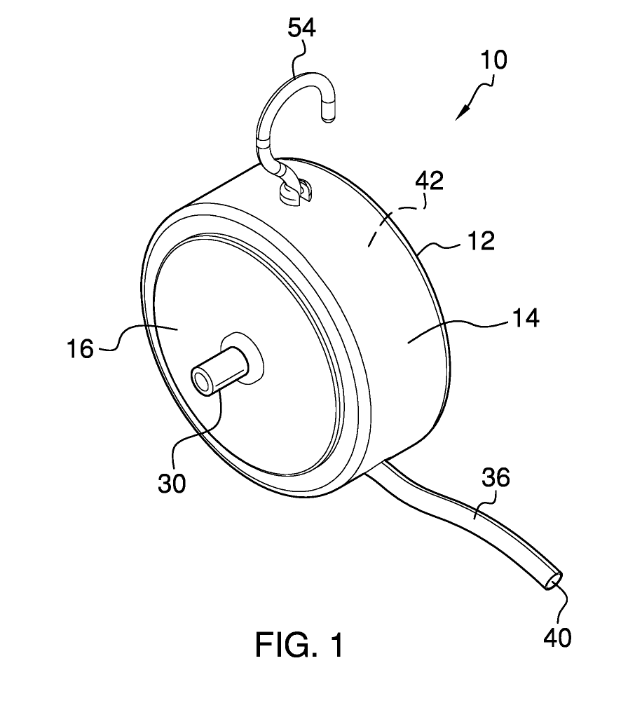

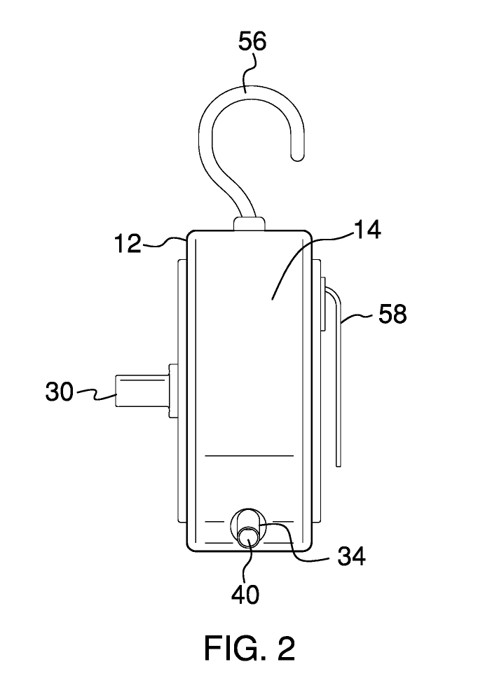

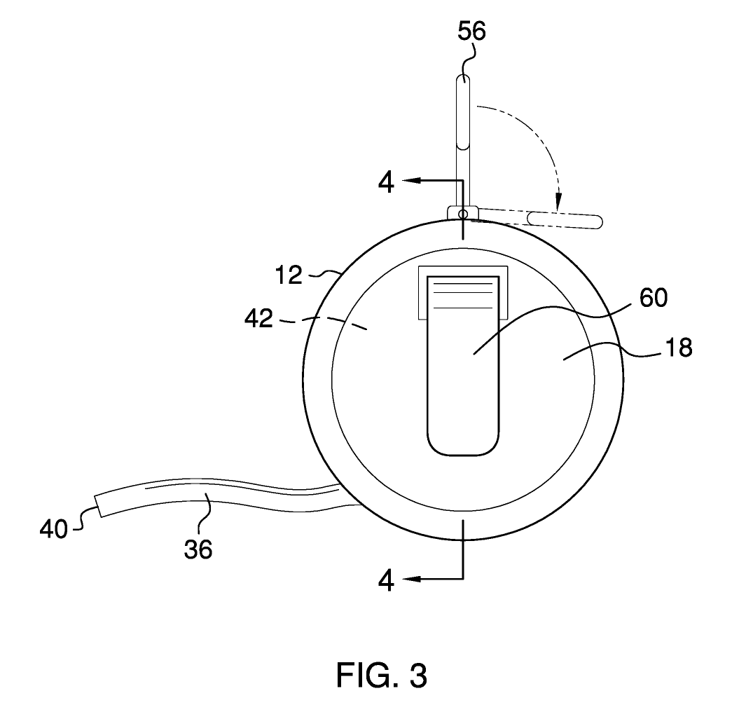

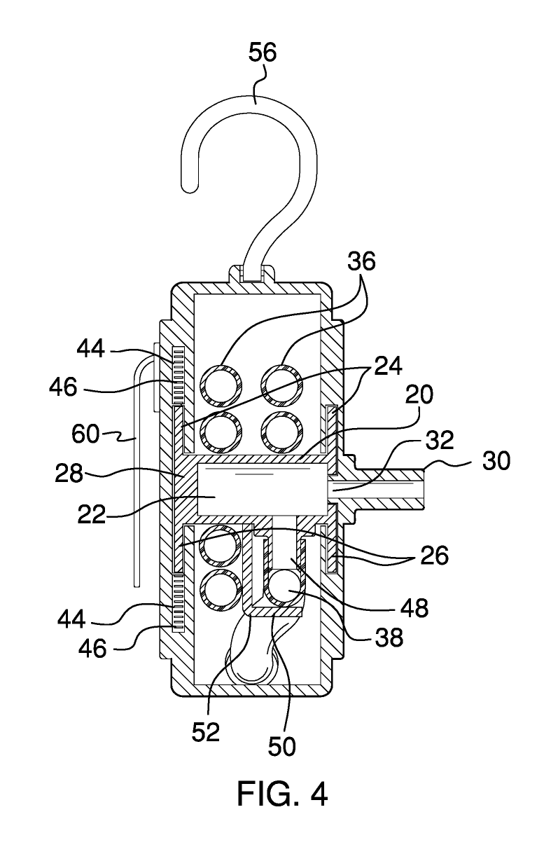

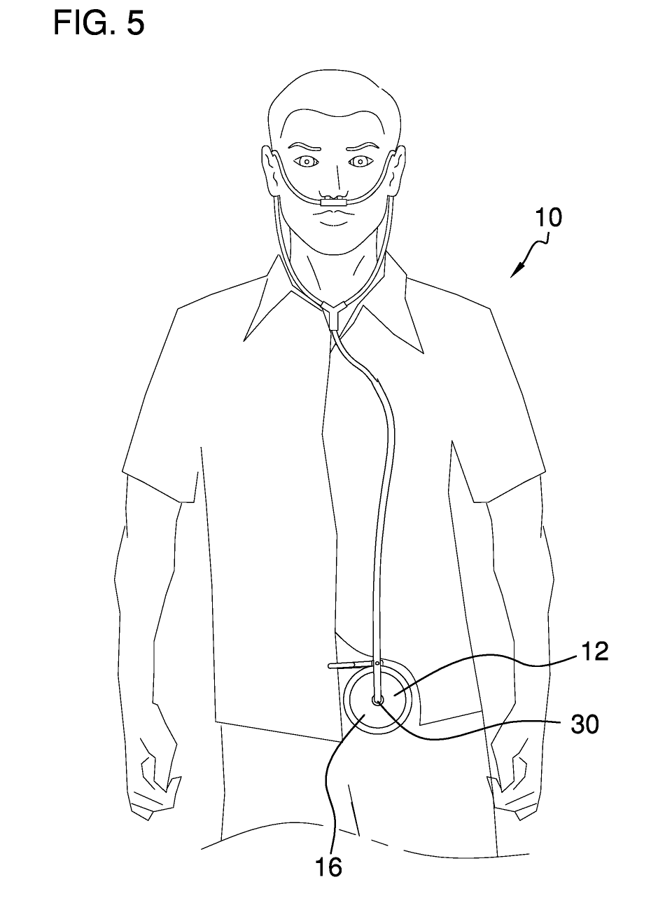

With reference now to the drawings, and in particular to FIGS. 1 through 5 thereof, a new retractable tubing device embodying the principles and concepts of an embodiment of the disclosure and generally designated by the reference numeral 10 will be described.

As best illustrated in FIGS. 1 through 5, the retractable tubing device 10 generally comprises a housing 12 that defines an internal space 42. The housing 12 is configured to couple to a user. In one embodiment, the housing 12 comprises an annular wall 14 that extends between a first side 16 and a second side 18 of the housing 12. In another embodiment, the first side 16 and the second side 18 are circularly shaped such that the housing 12 is cylindrically shaped.

A cylinder 20 is rotationally coupled to the housing 12 and is positioned in the internal space 42. The cylinder 20 extends between the first side 16 and the second side 18 of the housing 12 to define a chamber 22. The cylinder 20 is centrally positioned within the housing 12. The cylinder 20 is positioned to rotate relative to the housing 12.

A pair of first recesses 24 is positioned singly in the first side 16 and the second side 18 of the housing 12. Each of a pair of discs 26 is coupled to and extends from a respective opposing end 28 of the cylinder 20. Each disc 26 is complementary to and is positioned in a respective first recess 24. The discs 26 are positioned to rotate within the first recesses 24. The cylinder 20 is rotationally coupled to the housing 12 and is positioned to rotate relative to the housing 12.

A nozzle 30 is coupled to the housing 12. The nozzle 30 is fluidically coupled to the chamber 22. The nozzle 30 is configured to couple to tubing that is fluidically coupled to an oxygen supplying interface, such as a nasal cannula. In one embodiment, the nozzle 30 is centrally positioned on the first side 16 of the housing 12. A penetration 32 is positioned through a respective disc 26 proximate to the first side 16 of the housing 12. The nozzle 30 is aligned with the penetration 32.

An opening 34 is positioned in the housing 12. In one embodiment, the opening 34 is positioned in the annular wall 14.

The device 10 comprises a tube 36 that has a first end 38 and a second end 40. The first end 38 is fluidically coupled to the cylinder 20. The tube 36 extends through the opening 34. The tube 36 is flexible. The tube 36 is biased to a retracted configuration wherein the tube 36 is coiledly positioned around the cylinder 20 within the internal space 42. The tube 36 is selectively and reversibly extensible from the housing 12. The second end 40 of the tube 36 is configured to couple to a source of oxygen, such as an oxygen concentrator.

A second recess 44 extends from a respective first recess 24. A spring 46 is coupled to the housing 12 and is positioned in the second recess 44. The spring 46 is coupled to the disc 26 that is positioned in the respective first recess 24. The spring 46 is retractable flat coil wire styled. The spring 46 is positioned to rotate the cylinder 20 to coiledly position the tube 36 around the cylinder 20. The tube 36 is reversibly extensible from the housing 12 to allow oxygen to flow to the user when the user is positioned distal from the source of the oxygen, such as the oxygen concentrator.

A pipe 48 is fluidically coupled to and extends angularly from the cylinder 20. The pipe 48 is complementary to the tube 36. The pipe 48 is positioned to insert into the tube 36 to couple the tube 36 to the cylinder 20.

A connector 50 is coupled to the cylinder 20. The connector 50 is selectively couplable to the tube 36. The connector 50 is positioned to couple to the tube 36 to retain the tube 36 around the pipe 48. In one embodiment, the connector 50 comprises a bracket 52. The bracket 52 is flexible and L-shaped.

A coupler 54 is coupled to and extends from the annular wall 14. The coupler 54 is configured to couple the housing 12 to a mobility device, such as a wheelchair and a walker. In one embodiment, the coupler 54 comprises a hook 56. In another embodiment, the hook 56 is hingedly coupled to the housing 12. The hook 56 is selectively positionable in an extended arrangement to couple to the mobility device and a stowed arrangement when not in use.

A fastener 58 is coupled to the second side 18 of the housing 12. The fastener 58 is configured to couple the housing 12 to the user. In one embodiment, the fastener 58 comprises a belt clip 60. The belt clip 60 is configured to couple the housing 12 to a belt that is coupled to the user.

In use, the coupler 54 is configured to couple the housing 12 to the mobility device, such as the wheelchair and the walker. Alternatively, the belt clip 60 is configured to couple the housing 12 to the belt that is coupled to the user. The discs 26 that are positioned on the cylinder 20 are positioned to rotate within the first recesses 24. The cylinder 20 is rotationally coupled to the housing 12 and is positioned to rotate relative to the housing 12. The nozzle 30 that is positioned on the housing 12 is configured to couple to the tubing that is fluidically coupled to the oxygen supplying interface, such as the nasal cannula. The tube 36 is positioned through the opening 34 so that the second end 40 of the tube 36 is configured to couple to the source of the oxygen, such as the oxygen concentrator. The spring 46 is positioned to rotate the cylinder 20 to coiledly position the tube 36 around the cylinder 20. The tube 36 is reversibly extensible from the housing 12 to allow the oxygen to flow to the user when the user is positioned distal from the source of the oxygen, such as the oxygen concentrator.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A retractable tubing device comprising:
a housing defining an internal space, said housing being configured for coupling to a user;
a cylinder rotationally coupled to said housing and positioned in said internal space, said cylinder extending between a first side and a second side of said housing defining a chamber, said cylinder being centrally positioned within said housing;
a nozzle coupled to said housing, said nozzle being fluidically coupled to said chamber;
an opening positioned in said housing;
a tube having a first end and a second end, said first end being fluidically coupled to said cylinder, said tube extending through said opening, said tube being flexible, said tube being biased to a retracted configuration wherein said tube is coiledly positioned around said cylinder within said internal space; and
wherein said cylinder is positioned in said housing such that said cylinder is positioned for rotating relative to said housing, wherein said nozzle is positioned on said housing such that said nozzle is configured for coupling to tubing fluidically coupled to an oxygen supplying interface, such as a nasal cannula, wherein said tube is positioned on said cylinder such that said tube is selectively and reversibly extensible from said housing such that said second end of said tube is configured for coupling to a source of oxygen, such as an oxygen concentrator.

2. The device of claim 1, further including said housing comprising an annular wall extending between said first side and said second side, said first side and said second side being circularly shaped such that said housing is cylindrically shaped.

3. The device of claim 2, further comprising:
a pair of first recesses, said first recesses being positioned singly in said first side and said second side of said housing;
a pair of discs, each said disc being coupled to and extending from a respective opposing end of said cylinder, each said disc being complementary to and positioned in a respective said first recess;
a penetration positioned through a respective said disc proximate to said first side of said housing; and
wherein said discs are positioned on said cylinder such that said discs are positioned for rotating within said first recesses such that said cylinder is rotationally coupled to said housing and positioned for rotating relative to said housing.

4. The device of claim 3, further including said nozzle being centrally positioned on said first side of said housing such that said nozzle is aligned with said penetration.

5. The device of claim 2, further including said opening being positioned in said annular wall.

6. The device of claim 3, further comprising:
a second recess, said second recess extending from a respective said first recess;
a spring coupled to said housing and positioned in said second recess, said spring being coupled to said disc positioned in said respective said first recess, said spring being retractable flat coil wire styled; and
wherein said spring is positioned on said housing such that said spring is positioned for rotating said cylinder for coiledly positioning said tube around said cylinder such that said tube is reversibly extensible from said housing for allowing oxygen to flow to the user when the user is positioned distal from the source of the oxygen, such as the oxygen concentrator.

7. The device of claim 1, further including a pipe fluidically coupled to and extending angularly from said cylinder, said pipe being complementary to said tube, wherein said pipe is positioned on said cylinder such that said pipe is positioned for inserting into said tube for coupling said tube to said cylinder.

8. The device of claim 7, further including a connector coupled to said cylinder, said connector being selectively couplable to said tube, wherein said connector is positioned on said cylinder such that said connector is positioned for coupling to said tube for retaining said tube around said pipe.

9. The device of claim 8, further including said connector comprising a bracket, said bracket being flexible and L-shaped.

10. The device of claim 2, further including a coupler coupled to and extending from said annular wall, said coupler being configured for coupling said housing to a mobility device, such as a wheelchair and a walker.

11. The device of claim 10, further including said coupler comprising a hook.

12. The device of claim 11, further including said hook being hingedly coupled to said housing such that said hook is selectively positionable in an extended arrangement for coupling to the mobility device and a stowed arrangement when not being used.

13. The device of claim 2, further including a fastener coupled to said second side of said housing, said fastener being configured for coupling said housing to the user.

14. The device of claim 13, further including said fastener comprising a belt clip, wherein said belt clip is positioned on said housing such that said belt clip is configured for coupling said housing to a belt coupled to the user.

15. A retractable tubing device comprising:
a housing defining an internal space, said housing being configured for coupling to a user, said housing comprising an annular wall extending between a first side and a second side, said first side and said second side being circularly shaped such that said housing is cylindrically shaped;
a cylinder rotationally coupled to said housing and positioned in said internal space, said cylinder extending between said first side and said second side of said housing defining a chamber, said cylinder being centrally positioned within said housing, wherein said cylinder is positioned in said housing such that said cylinder is positioned for rotating relative to said housing;
a pair of first recesses, said first recesses being positioned singly in said first side and said second side of said housing;
a pair of discs, each said disc being coupled to and extending from a respective opposing end of said cylinder, each said disc being complementary to and positioned in a respective said first recess, wherein said discs are positioned on said cylinder such that said discs are positioned for rotating within said first recesses such that said cylinder is rotationally coupled to said housing and positioned for rotating relative to said housing;
a penetration positioned through a respective said disc proximate to said first side of said housing;
a nozzle coupled to said housing, said nozzle being fluidically coupled to said chamber, wherein said nozzle is positioned on said housing such that said nozzle is configured for coupling to tubing fluidically coupled to an oxygen supplying interface, such as a nasal cannula, said nozzle being centrally positioned on said first side of said housing such that said nozzle is aligned with said penetration;
an opening positioned in said housing, said opening being positioned in said annular wall;
a tube having a first end and a second end, said first end being fluidically coupled to said cylinder, said tube extending through said opening, said tube being flexible, said tube being biased to a retracted configuration wherein said tube is coiledly positioned around said cylinder within said internal space, wherein said tube is positioned on said cylinder such that said tube is selectively and reversibly extensible from said housing such that said second end of said tube is configured for coupling to a source of oxygen, such as an oxygen concentrator;
a second recess, said second recess extending from a respective said first recess;
a spring coupled to said housing and positioned in said second recess, said spring being coupled to said disc positioned in said respective said first recess, said spring being retractable flat coil wire styled, wherein said spring is positioned on said housing such that said spring is positioned for rotating said cylinder for coiledly positioning said tube around said cylinder such that said tube is reversibly extensible from said housing for allowing oxygen to flow to the user when the user is positioned distal from the source of the oxygen, such as the oxygen concentrator;
a pipe fluidically coupled to and extending angularly from said cylinder, said pipe being complementary to said tube, wherein said pipe is positioned on said cylinder such that said pipe is positioned for inserting into said tube for coupling said tube to said cylinder;
a connector coupled to said cylinder, said connector being selectively couplable to said tube, wherein said connector is positioned on said cylinder such that said connector is positioned for coupling to said tube for retaining said tube around said pipe, said connector comprising a bracket, said bracket being flexible and L-shaped;
a coupler coupled to and extending from said annular wall, said coupler being configured for coupling said housing to a mobility device, such as a wheelchair and a walker, said coupler comprising a hook, said hook being hingedly coupled to said housing such that said hook is selectively positionable in an extended arrangement for coupling to the mobility device and a stowed arrangement when not being used;
a fastener coupled to said second side of said housing, said fastener being configured for coupling said housing to the user, said fastener comprising a belt clip, wherein said belt clip is positioned on said housing such that said belt clip is configured for coupling said housing to a belt coupled to the user; and
wherein said discs are positioned on said cylinder such that said discs are positioned for rotating within said first recesses such that said cylinder is rotationally coupled to said housing and positioned for rotating relative to said housing, wherein said nozzle is positioned on said housing such that said nozzle is configured for coupling to the tubing fluidically coupled to the oxygen supplying interface, such as the nasal cannula, wherein said tube is positioned through said opening such that said second end of said tube is configured for coupling to the source of the oxygen, such as the oxygen concentrator, wherein said spring is positioned on said housing such that said spring is positioned for rotating said cylinder for coiledly positioning said tube around said cylinder such that said tube is reversibly extensible from said housing for allowing oxygen to flow to the user when the user is positioned distal from the source of the oxygen, such as the oxygen concentrator.
